(19) United States
(12) Patent Application Publication
(10) Pub. No.: US 2007/0280973 A1
(43) Pub. Date: Dec. 6, 2007

(54) Composite material and method of producing a composite material of this type

(76) Inventors: Arno Schmuck, Leichlingen (DE); Jorg Siegel, Koln (DE); Christian Schonefeld, Geilenkirchen (DE)

Correspondence Address: WILLIAM COLLARD;COLLARD & ROE, P.C., 1077 NORTHERN BOULEVARD, ROSLYN, NY, 11576, US

(21) Appl. No.: 11/809,620

(22) Filed: Jun. 1, 2007

Related U.S. Application Data
(60) Provisional application No. 60/811,981, filed on Jun. 8, 2006.

(30) Foreign Application Priority Data
Jun. 2, 2006 (DE) 10 2006 026 302.2
Sep. 11, 2006 (DE) 10 2006 043 216.9

(51) Int. Cl. A61K 9/00

(52) U.S. Cl. 424/400

(57) ABSTRACT

To refine composite materials, the present invention suggests a composite material made of at least three layers, in which at least one of the layers has active ingredient, ceramic nanoparticles, silver salts, or nanoparticulate carbon modifications.

The present invention relates on one hand to a composite material made of at least three layers and on the other hand to a method for producing a composite material having at least three layers, in which the layers are cast on a carrier material.

Composite materials have been known for some time from the prior art. Especially good properties which are intrinsic to individual materials may be linked with one another on a single component, i.e., a component made of a composite material, and used via composite materials of this type.

It is the object of the present invention to provide composite materials, using which new areas of application may be opened up.

The object of the present invention is achieved by a composite material made of at least three layers, at least one of the layers having active ingredients, ceramic nanoparticles, silver salts, or nanoparticulate carbon modifications.

In the present case, the term “layers” includes individual material regions of the composite material which form a complex composite material layered one on top of another as layers.

It is obvious that in the meaning of the present patent application, the composite material may have nearly arbitrarily many layers of this type. However, at least three layers are typically necessary for the composite material according to the present invention, so that in at least one area, one of the layers is completely covered by the further layers on at least two of its main sides. However, the present invention also relates to composite materials having fewer layers, if the composite materials have the cited active ingredients, nanoparticles, silver salts, or carbon modifications.

The object of the present invention is also achieved by a composite material made of at least three layers, the composite material being produced using a cascade casting machine or a curtain casting machine. Cascade casting or curtain casting advantageously allows the application of multiple layers, also of different thicknesses, onto a carrier material in one work step. The present composite material may thus be produced using a low construction outlay.

According to the method, the object of the present invention is achieved by a method for producing a composite material having at least three layers, in which the layers are cast on a carrier material, which is distinguished in that at least one further layer admixed with a component different from the first component is cast onto a layer admixed with a first component.

Composite materials whose layers have different functionalities may be produced via the casting of the different layers on one another.

The composite material on which the present invention is based has the further advantage in relation to known composite materials that in the present case layers may be achieved having a significantly higher precision in regard to the layer thickness. In the prior art, the layer thicknesses have a tolerance of ±10%. In the composite material according to the present invention, the individual layer thicknesses have a tolerance below ±10%. Tolerances of ±1% are even achievable for the layer thicknesses of the individual composite material layers.

In a preferred embodiment variation of the present composite material, at least one of the layers has medicinal active ingredients or bitter principles.

Providing a single-layer film which is water-soluble, for example, with medicinal active ingredients is known from the prior art. The medicinal active ingredients are gradually released as the single-layer film dissolves. However, many medicinal active ingredients have an unpleasant taste to a user who has to consume a medicinal active ingredient orally, so that oral administration of medicinal active ingredients is usually problematic.

In particular for animals, administering medicinal active ingredients is frequently necessary, because domestic and utility animals have to be dewormed regularly, for example. Active ingredients which predominantly act against tapeworms are orally consumed by cats and dogs only very unwillingly. In particular the active ingredient praziquantel is an especially bitter-tasting active ingredient, which is not orally consumed willingly by animals. The introduction of the active ingredients into pastes or tablets does improve the willingness to consume, but does not yet lead to satisfactory results, above all for cats.

Surprisingly, it has been found that using the present composite material, for example, a bad-tasting active ingredient may be embedded in an extremely thin layer, also in high concentration, the thin layer having the bad-tasting active ingredient being covered and encapsulated by further layers. The further layers may advantageously have a pleasant-smelling and/or pleasant-tasting active ingredient in the present case, so that the thin layer having the bad-tasting active ingredient is particularly advantageously embedded in the composite material.

In the future, there will increasingly be oral applications for self-medication. Many medicinal active ingredients in medications are unpleasant in taste or smell, however. As a result, it is particularly advantageous if the layer having the active ingredients is a middle layer in the composite material, which is enclosed by at least two further layers.

In particular when the present composite material having a first layer containing a medicinal active ingredient is produced according to the present invention using a cascade method, the encapsulation of the first layer is implemented very simply.

Active ingredients in the composite material which are perceivable as unpleasant may additionally be masked if at least one of the layers has flavoring substances. Flavoring substances of this type are advantageously provided in layers which encapsulate the layer containing the active ingredients.

To make it easier to administer active ingredients which are contained in the present composite material to living beings, it is advantageous if the composite material has a cylindrical body. For this purpose, the layer containing the active ingredients is carefully wound up, so that the composite material itself may be chewed without directly damaging the layer containing active ingredients.

In a further preferred exemplary embodiment, at least one of the layers has ceramic nanoparticles. Thin ceramic films may also be produced using a composite material of this type. In particular if the at least three-layered composite material having a layer having ceramic nanoparticles is produced using a cascade casting method or a curtain casting method, significantly thinner ceramic films may be produced than known from the prior art. Films of this type have usually been produced up to this point using a doctor blade production method. However, this is complex and significantly less thin films may thus be produced.

In particular, the films produced according to the present invention are distinguished by especially high uniformity of the layers. Therefore, films of extremely high quality are producible rapidly and cost-effectively.

It is therefore advantageous in connection with the composite material on which the present invention is based if the composite material comprises a film, in particular a ceramic film.

In a further advantageous embodiment, at least one of the layers has sensitized silver salts. In a particularly advantageous embodiment in this regard, the sensitized silver salts are made electrically conductive using a chemical reduction. Therefore, electrical conductors are implemented particularly advantageously using the present composite material.

Also in an advantageous embodiment variation, one of the layers has carbon fullerene. For example, photoconductive films may advantageously be produced using the three-layered composite material according to the present invention. A technical area of application for films of this type is particularly in photocopying devices.

In the present case, fullerenes, in particular carbon fullerenes, are advantageously embedded in a polymer.

All above-mentioned composite material variants made of at least three layers advantageously share the feature that at least one of the layers has first components which are different from further components of further layers.

Therefore, in a further embodiment variation, the layer which has at least one component, namely active ingredients, in particular medicinal active ingredients or bitter principles, ceramic nanoparticles, silver salts, in particular sensitized silver salts, or nanoparticulate carbon modifications, in particular carbon fullerenes, is enclosed by at least two further layers.

In order to be able to cover a first layer of the composite material on both sides with further layers and thus encapsulate it from the environment, it is advantageous if at least two layers have identical properties.

In addition, it is advantageous if at least two layers have identical components. In particular if the one first layer having first components is enclosed on both sides by a further layer having identical components in each case, a symmetrical composite material is implemented.

It is obvious that the layers of the present composite material may have relatively high thicknesses. In particular, the layer thicknesses achievable using a known cascade and/or curtain casting method may be implemented. However, to be able to construct the composite material as compactly as possible, it is advantageous if at least one of the layers has a layer thickness of less than 20 μm or less than 0.5 μm. Especially thin films may thus also be implemented using the present composite material.

It has been found that a sufficiently large quantity of components such as medicinal active ingredients may be incorporated even in extremely thin composite material layers. To also be able to incorporate larger quantities of components in individual layers of the composite material, it is advantageous if at least one of the layers has a layer thickness of more than 0.1 μm or more than 0.4 μm.

Components of one of the layers may be implicated in one of the layers with an especially simple construction if at least one of the layers has gelatin.

In a further composite material variant, at least one of the layers has a ceramic suspension having less than 80 wt.-%, preferably less than 70 wt.-% ceramic nanoparticles.

In addition, it is advantageous if at least one of the layers has a ceramic suspension having more than 30 wt.-%, preferably more than 45 wt.-% ceramic nanoparticles.

A quantity of ceramic nanoparticles in the above-mentioned boundaries is favorable for an advantageous implementation of ceramic films.

If one layer of the present composite material has ceramic nanoparticles, it is advantageous if the ceramic nanoparticles have a grain size of less than 5000 nm, preferably of less than 1000 nm.

In addition, it is advantageous if the ceramic nanoparticles have a grain size of more than 10 nm, preferably more than 100 nm.

However, areas of application are known, in connection with fuel cells, for example, in which films having extremely small pore sizes must be implemented. It has been shown here that nanoparticles having grain sizes between 1 nm and 100 nm must be used, so that grain sizes of this type may also be advantageous in the present case.

To stabilize components which are contained in a layer in this layer, it is advantageous if at least one of the layers has a thickener, a hardening agent for a component of a layer, such as gelatin, or a cross-linking agent for a component of a layer, such as gelatin.

It is advantageous if one of the layers is an electrically functionalized film. Using the composite material according to the present invention, very thin electrical conductors may advantageously be provided having an especially simple construction.

In particular if the composite material is electrically conductive, i.e., it is to form and/or provide an electrical conductor, is advantageous if at least one of the layers is electrically conductive.

An especially high-quality electrically conductive composite material is provided if at least one electrically conductive layer has silver and/or silver salt. In the present case, silver chloride, silver bromide, silver iodide, or mixed forms thereof may preferably be used as the silver salt.

An electrically insulated conductor in the form of the present composite material is provided if an electrically conductive layer is enclosed by electrically insulating layers.

It is also advantageous if the electrically insulating layers have a polymer, such as gelatin, as the insulator. It is obvious that besides a polymer, any other materials may also be used as an insulator, as long as they are capable of forming an insulating layer of the present composite material.

If the composite material is to have one or more layers having silver salts, it is advantageous if the silver salts are sensitized in a range above 300 nm, preferably above 350 nm.

In addition, it is advantageous if the silver salts are sensitized in a range below 800 nm, preferably below 750 nm.

The advantages of the silver halogenide salts sensitized to different wavelength ranges, for example, for the present composite material are that different printed conductor structures may advantageously be exposed in different layers in one step. In this case, the printed conductor grids are equipped with different sensitizers for specific wavelength ranges and preferably exposed using light of different wavelengths. For example, a printed conductor grid A in a layer X is exposed using light of the wavelength 380 nm to 480 nm (blue light), and a printed conductor grid B in a further layer Y is exposed using light of the wavelength 590 nm to 800 nm (red light). Two differentiable printed conductor grids arise simultaneously in a subsequent chemical processing.

In an embodiment variation in this regard, the silver salts are fixed using ammonium thiosulfate or sodium thiosulfate.

To produce composite materials which are specially tailored for specific applications, it is advantageous if at least two layers have layer thicknesses different from one another.

To produce the present composite material, but also for the further handling of this composite material comprising at least three layers, it is advantageous if at least one of the layers is situated on a carrier material. It is specific to the application in the present case whether the composite material remains situated on the carrier material or is pulled off of it before the actual use.

In order that the composite material is not or is only slightly deformed during the production of the composite material, in particular during a firing procedure of individual films of the composite material, it is advantageous if the composite material is implemented symmetrically.

In particular if a composite material is a soluble film, it is advantageous if polymer-containing layers comprise a water-soluble polymer.

Further advantages, goals, and properties of the present invention are described on the basis of the drawing appended to the following explanation, in which different composite materials and their areas of application as well as their compositions are described for exemplary purposes.

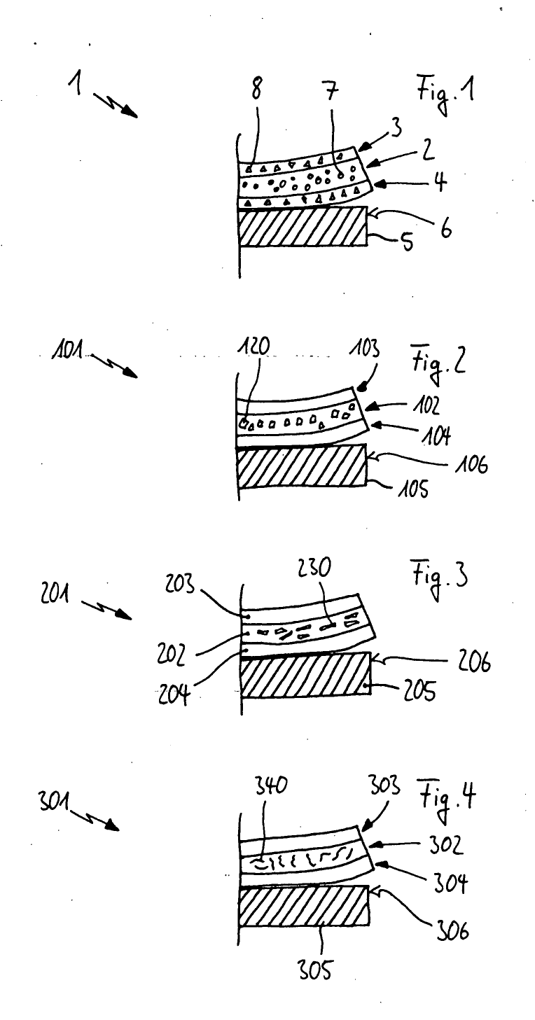

FIG. 1 schematically shows a longitudinal section through a composite material on a carrier layer, a first layer admixed with medicinal active ingredients being enclosed by a second layer and a third layer, which have flavoring substances,

FIG. 2 schematically shows a longitudinal section through a further composite material on a carrier layer, a first layer admixed with ceramic nanoparticles being enclosed by a second layer without ceramic nanoparticles of this type and a third layer without ceramic nanoparticles of this type,

FIG. 3 schematically shows a longitudinal section through an alternative composite material on a carrier layer, a first layer admixed with a spectrally sensitized silver salt being enclosed by a second layer and a third layer made of a nonconductive polymer, and

FIG. 4 schematically shows a longitudinal section through a further composite material on a carrier layer, a first layer admixed with nanocarbon fullerenes being enclosed by a second layer without nanocarbon fullerenes of this type and a third layer without nanocarbon fullerenes of this type.

The composite material 1 shown in FIG. 1 comprises a middle layer 2, an upper layer 3, and a lower layer 4, which is situated on a carrier material 5. In the forward area 6 of the composite material 1, the lower layer 4 is already somewhat detached from the carrier material 5.

In this exemplary embodiment, the middle layer 2 comprises components of a medicinal active ingredient 2 and is completely enclosed by the upper and lower layers 3 and 4.

Both the upper layer 3 and also the lower layer 4 comprise flavoring substances 8, which are pleasant for a sensory perception of an animal in this exemplary embodiment.

All three layers 2, 3, and 4 of the composite material 1 are melted to one another. The three layers 2, 3, and 4 of the present composite material 1 were joined to one another using a known cascade casting method.

Because of the fact that the middle layer 2 having its medicinal active ingredient 1 is completely encapsulated using the upper and lower layers 3 and 4 comprising the flavoring substances 8, the present composite material 1 is outstandingly suitable for oral administration of a medicinal active ingredient 7, in particular to an animal.

As noted at the beginning, there will increasingly be oral applications for self-medication. Many active ingredients in medications are unpleasant in taste or smell. Surprisingly, it has been found that a composite material 1 produced using a cascade casting method may contain a bad-tasting medicinal active ingredient 7 in extremely thin layers 2 in high concentration and nonetheless be effectively concealed in taste using multiple layers 3, 4.

The degradation of the good-tasting layers 3, 4 in the meaning of the present patent application may be controlled by the selection of the matrix, which may be built up from natural materials such as gelatin, cellulose, or chitins, and the selection of a suitable cross-linking agent (hardener) in such a way that even in the event of longer chewing or licking, the medicinal active ingredient 7 is not released in the mouth or on the taste organs.

A cast assembly comprising at least three layers 2, 3, and 4 [A-B-A] is applied to a carrier 5 using a cascade casting machine, whose first and last layers 3 and 4 contain an odorant or a flavoring substance 8, which may be introduced in different concentrations. This odorant or flavoring substance 8 is freely selectable. The middle layer 2 may contain the medicinal active ingredient 7. This structure (three layers) may be pulled (stripped) off of the carrier material 5 and assembled.

For example, pellets may be produced which contain a defined active ingredient quantity. Dosing of the stripped-off films via the available area of one of the layers 2, 3, and/or 4 would also be possible. The medicinal active ingredient 7 is, for example, to be embedded in a gelatin composite in such a way that the medicinal active ingredient 7 is willingly consumed by an animal, for example, due to gelatin layers which mask the odor and taste, and the medicinal active ingredient 7 is first released in the stomach and not already when it is taken into the mouth, and thus highly willing consumption occurs.

It is obvious that the present composite material 1 may also advantageously be used for humans, in particular for children.

A casting solution for a layer 2, 3, and 4 may be produced as follows. The medicinal active ingredients 7 used are almost entirely insoluble in water and may also only be dissolved in alcohol in small quantities. Therefore, a suspension was manufactured in gelatin. To ensure uniform distribution of the medicinal active ingredients 7 and to pulverize coarser particles (praziquantel forms long needles, for example), the following procedure has proven itself:

[65] Gelatin is swollen in cold water and subsequently dissolved at 40° C. The active ingredient combination is added to the gelatin solution and stirred for three minutes at 3000 RPM using an Ultra-Turrax stirrer (stirring rod having a very rapidly rotating blade, up to 24,000 RPM), for example. Due to the high speed of revolution of the blade, high shear forces arise and particles in a suspension are chopped very small. The following ingredients are necessary in the present case:

I. Gelatin: pharmaceutical gelatin from Gelita
140 bloom,
160 bloom,
or 200 bloom.

The bloom value indicates the gelling power of a type of gelatin and is therefore also a parameter of the solubility in water.

II. Glycerin: glycerin is advantageously added to the casting solution as a softener, because the layer composite becomes very brittle without this additive and this would make further processing more difficult.

III. Substrate: because the layers must be able to be stripped off of the substrate, an unsubstituted polyester substrate is selected.

Formulation of Active Ingredient Combination for Cats (5 kg):

Combination: pyrantel embonate 59 mg/kg, γ=295 mg/5 kg, praziquantel 5 mg/kg, γ=25 mg/5 kg, sum: 320 mg active ingredient per dose unit.

Layer Construction:

| | Layer thickness wet |
|---|---|
| Gelatin | 180 μm |
| Active ingredient combination | 180 μm |
| Gelatin | 180 μm |

Total layer thickness after drying: 55 μm.

Recipe of Casting Solutions:

| | |
|---|---|
| Outer layers: | 250 g gelatin |
| | 2190 g water |
| | 60 g glycerin |
| | 2500 g casting solution |

| | |
|---|---|
| Active ingredient layer: | 125 g gelatin |
| | 1001 g water |
| | 30 g glycerin |
| | 86.43 g pyrental embonate |
| | 7.32 g praziquantel |
| | 1250 g casting solution |

A 230 cm² large film piece contains 320 mg of the active ingredient combination for a cat weighing 5 kg.

Formulation of Active Ingredient Combination for Dogs (15 kg):

Combination: pyrantel embonate 14.5 mg/kg, γ=217.5 mg/S kg, praziquantel 5 mg/kg, γ=75 mg/5 kg, febantel 15 mg/kg, γ=225 mg/5 kg, sum: 517.5 mg of active ingredient per dose unit.

In addition, a flavor (meat flavor) was incorporated into the outer layers. The incorporation was performed as described above using an Ultra-Turrax stirrer.

Layer Construction:

| | Layer thickness wet |
|---|---|
| Gelatin | 180 μm |
| Active ingredient combination | 180 μm |
| Gelatin | 180 μm |

Total layer thickness after drying: 55 μm.

Recipe of Casting Solutions:

| | |
|---|---|
| Outer layers: | 250 g gelatin |
| | 2178 g water |
| | 62.4 g glycerin |
| | 9.83 g flavor |
| | 2500 g casting solution |

| | |
|---|---|
| Active ingredient layer: | 125 g gelatin |
| | 1001 g water |
| | 31.2 g glycerin |
| | 39.4 g pyrental embonate |
| | 13.58 g praziquantel |
| | 40.76 g febantel |
| | 1250 g casting solution |

A 372.15 cm² large film piece contains 517.5 mg of the active ingredient combination for a dog weighing 15 kg.

Pieces were cut out of the stripped-off film (preferably having a precisely defined active ingredient quantity). These active ingredient quantities were able to be incorporated into wet food shredded or whole. The gelatin does swell in a damp environment, but holds the medicinal active ingredients 7 in the solid composite. Oblong pieces were cut out of the film, which were subsequently rolled up so that a cylindrical shape resulted. The following cylinders were implemented:

| dog (15 kg) | cat (per kilogram of body weight) |
|---|---|
| d = 1.1 cm | d = 0.6 cm |
| h = 2.4 cm | h = 1.2 cm |

The following advantageous results were able to be achieved in field tests which were performed for dogs in comparison to administration forms up to this point:

| | Administration forms | | | |
|---|---|---|---|---|
| | | | | Film |
| | | | Film (processed | administered |
| | Paste | Tablet | further as a roll) | in soft food |
| % willing consumption | 60 | 68 | 95 | 98 |

The following advantageous results ere able to be achieved in field tests which were performed for cats in comparison to administration forms up to this point:

| | Administration forms | | | |
|---|---|---|---|---|
| | | | | Film |
| | | | Film (processed | administered |
| | Paste | Tablet | further as a roll) | in soft food |
| % willing consumption | 55 | 54 | 90 | 94 |

The composite material 101 shown in FIG. 2 comprises a middle layer 102, an upper layer 103, and a lower layer 104, which is situated on a carrier material 105. In the forward area 106 of the composite material 101, the lower layer 104 is already somewhat detached from the carrier material 105.

In this exemplary embodiment, the middle layer 102 comprises ceramic nanoparticles 120 and is completely enclosed by the upper and lower layers 103 and 104, which do not comprise ceramic nanoparticles 120 of this type. The upper and lower layers 103, 104 are based on a gelatin.

All three layers 102, 103, and 104 of the composite material 101 are melted to one another. The three layers 102, 103, and 104 of the present composite material 101 were joined to one another using a known cascade casting method.

In particular, significantly thinner ceramic films than in the prior art may be produced via the use of cascade or curtain casting machines and/or the uniformity of the layers 102, 103, 104 may be significantly improved.

For this purpose, the ceramic nanoparticles 120 are preferably introduced into a casting solution based on gelatin. Surprisingly, it has also been found that up to 10 layers provided with ceramic nanoparticles may be applied in a single work step to a carrier as differently functionalized individual layers, which may also contain different ceramic particles in different grain sizes and concentrations, preferably if a cascade or curtain casting method is used for this purpose.

The layers 103 and 104 implemented as films may be constructed symmetrically or asymmetrically around a functional middle layer 102. A symmetrical structure [A-B-C-D-C-B-A] has the advantage that films of this type deform only very slightly or not at all during the firing procedure.

A further advantage is the possibility of being able to manufacture multilayered ceramic films which are distinguished by extremely thin layer thicknesses. The ceramic films (layers having the ceramic nanoparticles) are preferably produced, as described above, in the cascade casting method.

In general, an unsubstituted polyester web may advantageously be selected as the substrate (carrier layer 105), because the films are stripped off of the substrate.

In this exemplary embodiment, a thin gelatin layer is advantageously cast as the first layer, which makes the stripping easier.

Gelatin Recipe:

100 g gelatin

890 g water

10 g glycerin

1000 g casting solution

I. Films having aluminum oxide nanoparticles:

Casting Solution 1:

| | Quantity (g) |
|---|---|
| Aluminum oxide d₅₀ = 1 μm | 3800 |
| 5% gelatin | 1600 |
| Liquefier | 8 |
| Wetting agent | 75 |
| Glycerin | 30 |
| Sum | 5,438 |

Casting Solution 2:

| | Quantity (g) |
|---|---|
| Aluminum oxide d₅₀ = 2.5 μm | 3800 |
| 5% gelatin | 1600 |
| Liquefier | 8 |
| Wetting agent | 75 |
| Glycerin | 30 |
| Sum | 5,438 |

Casting Solution 3:

| | Quantity (g) |
|---|---|
| Aluminum oxide d₅₀ = 4 μm | 3800 |
| 5% gelatin | 1600 |
| Liquefier | 8 |
| Wetting agent | 75 |
| Glycerin | 30 |
| Sum | 5,438 |

Layer Structure 1:

| | Layer thickness wet |
|---|---|
| Gelatin (10%) | 30 μm |
| Casting solution 1 | 60 μm |
| Casting solution 2 | 60 μm |
| Casting solution 3 | 60 μm |

dry layer thickness: 158 μm

Layer Structure 2:

| | Layer thickness wet |
|---|---|
| Gelatin (10%) | 30 μm |
| Casting solution 1 | 60 μm |
| Casting solution 2 | 60 μm |
| Gelatin (10%) | 30 μm |

dry layer thickness: 92.04 μm

Layer Structure 3:

| | Layer thickness wet |
|---|---|
| Gelatin (10%) | 30 μm |
| Casting solution 1 | 60 μm |

dry layer thickness: 46.02 μm

Layer Structure 4:

| | Layer thickness wet |
|---|---|
| Gelatin (10%) | 10 μm |
| Casting solution 3 | 20 μm |

dry layer thickness: 15.34 μm

II. Films Made of Zirconium Oxide:

Casting Solution 1:

| | Quantity (g) |
|---|---|
| Zirconium oxide d₅₀ = 1.3 μm | 3150 |
| 10% gelatin | 1850 |
| Liquefier | 6.5 |
| Wetting agent | 40 |
| Glycerin | 25 |
| Sum | 5,071.5 |

Casting Solution 2:

| | Quantity (g) |
|---|---|
| Zirconium oxide d₅₀ = 3.5 μm | 3150 |
| 10% gelatin | 1850 |
| Liquefier | 6.5 |
| Wetting agent | 40 |
| Glycerin | 25 |
| Sum | 5,071.5 |

Layer Structure 1:

| | Layer thickness wet |
|---|---|
| Gelatin (10%) | 20 μm |
| Casting solution 1 | 20 μm |
| Casting solution 2 | 40 μm |

dry layer thickness: 39.8 μm

Layer Structure 2:

| | Layer thickness wet |
|---|---|
| Gelatin (10%) | 30 μm |
| Casting solution 1 | 20 μm |

dry layer thickness: 15.6 μm

III. Films Made of Silicon Carbide:

Casting Solution 1:

| | Quantity (g) |
|---|---|
| Zirconium oxide d₅₀ = 0.8 μm | 2900 |
| 5% gelatin | 1700 |
| Liquefier | 8 |
| Wetting agent | 35 |
| Glycerin | 30 |
| Sum | 4,673 |

Layer Structure 1:

| | Layer thickness wet |
|---|---|
| Gelatin (10%) | 10 μm |
| Casting solution 1 | 20 μm |

dry layer thickness: 13.8 μm

Layer Structure 2:

| | Layer thickness wet |
|---|---|
| Gelatin (10%) | 10 μm |
| Casting solution 1 | 10 μm |

dry layer thickness: 6.4 μm

The technology according to the present invention provides the production advantages summarized in the following table:

| | Production | Thickness |
|---|---|---|
| | thickness | tolerances |
| Prior art | >30 μm | 50 μm ± 5 μm |
| Technique according to the present invention | >5 μm | 50 μm ± 1 μm |

The composite material 201 shown in FIG. 3 comprises a middle layer 202, an upper layer 203, and a lower layer 204, which is situated on a carrier material 205. In the forward area 206 of the composite material 201, the lower layer 204 is already somewhat detached from the carrier material 205.

The middle layer 202 is electrically conductive in this exemplary embodiment and is completely enclosed by the upper electrically nonconductive layer 203 and the lower electrically nonconductive layer 204. The upper and lower layers 203, 204 are based on a gelatin. The middle layer 202 contains a spectrally sensitized silver salt 230 in this exemplary embodiment.

All three layers 202, 203, and 204 of the composite material 201 are melted to one another. The three layers 202, 203, and 204 of the present composite material 201 were joined to one another using a known cascade casting method.

An advantageous electrical conductor having an electrically nonconductive polymer is provided via the present composite material 201. This polymer is gelatin in the present case.

Surprisingly, it has been found that in a layer composite comprising three layers 202, 203, and 204 [A-B-C], electrical conductors, which are distinguished by a high degree of separation, may be generated by exposure, reduction, and fixing. The layers A and C are typically not electrically conductive and comprise a polymer, such as gelatin. A middle layer B contains a spectrally sensitized silver salt, such as silver chloride, silver bromide, or silver iodide, or mixed forms thereof. Elementary silver may be precipitated by exposure and subsequent photographic analog reduction, for example, using a hydroquinone or ascorbic acid. The elementary silver thus precipitated is still located in a silver salt environment, which may advantageously be dissolved out of the layer composite (composite material 201) using a fixation having potassium or sodium or ammonium thiosulfate. For example, it is known from photographic materials that gelatin represents a protective colloid for silver salts, which stabilizes the silver salt used here.

Due to a sensitization of the silver halogenide nanoparticles designed for various wavelength ranges of the light, it is possible to generate differently defined printed conductors simultaneously. For example, in a layer sequence [A-B-C-D-C-E-A], three different electric circuits may be implemented in the layers B, D, and E. These conductors containing silver are distinguished by better conductivity than in the prior art, in addition to the greater variability in the multilayered structuring.

For example, a photographic recording material, which is suitable for a rapid processing process, is produced by applying the following layers in the specified sequence on a paper coated with polyethylene on both sides.

The following quantity specifications relate in each case to 1 m². For the silver halogenide application, the corresponding quantities of AgNO₃ are specified in g/m².

I. Production of the Silver Halogenide Emulsion:

Solution 1:

6000 g demineralized water

180 g gelatin

10 g NaCl

14 ml sulfuric acid (25 wt.-%)

Solution 2:

1400 g demineralized water

57 g NaCl

112 g KBr

Solution 3:

1400 g demineralized water

320 g AgNO₃

Solution 4:

1800 g demineralized water

132 g NaCl

238 g KBr

0.4 mg K₂lrCl₆

0.076 mg RhCl₃

Solution 5:

1800 g demineralized water

680 g AgNO₃

Solution 1 is prepared and heated to 65° C. While maintaining this temperature, solutions 2 and 3 are added simultaneously within 35 minutes at a pAg value of 8 of solution 1. Solutions 4 and 5 are then added simultaneously in 45 minutes while maintaining pAg 8 at 65° C. A silver chloride-bromide emulsion having 50 mol-% each AgCl and AgBr having a mean particle diameter of 0.86 μm is obtained. The emulsion is flocculated, washed, and redispersed using enough gelatin that the gelatin/AgNO₃ weight ratio is 13. Subsequently, the solution is optimally ripened at a pH value of 4.5 using 3.4 micromole gold chloride per mole of silver and 0.7 micromole thiosulfate per mole of silver at 60° C.

II. Production of the Conductive Layers:

The photographic layer structures are applied to a polyester carrier of 175 μm thickness. The applications of the layer components are specified in g/m², if not otherwise noted.

In case of the silver halogenide emulsion, the AgNO₃ equivalent is specified as the application dimension. Example 1 and example 2 do not differ in the layer construction, but rather only in the selected wet-chemistry processing.

Layer Structure:

| First layer: 2.0 g gelatin | |
|---|---|
| Second layer: 2.0 g AgCl/Br, 550 μmol Sens-1 (in relation to | |
| moles Ag), 1.2 g gelatin | |
| Sens-1 [see source for chemical formula] | R₁ = C₂H₅ |
| | R₂ = R₃ = (CH₂)₄ |

Third layer: 2.0 g gelatin, 0.5 g hydroquinone, 0.025 g
benzotriazole, 0.05 g formalin

Performing the experiments:

A line raster having 4 lines/1 mm is exposed on the material, processed in the processing processes specified below, and the conductivity of a printed conductor of 4 lines is then measured.

### EXAMPLE 1

### Comparison

Developer

| | | |
|---|---|---|
| potassium sulfite solution, D = 1.45 | 375 | ml |
| l-phenyl-4-methyl-3-pyrazolidinone | 0.8 | g |
| phenidone | 0.5 | g |
| hydroquinone | 30.0 | g |
| potassium carbonate | 219.0 | g |
| ethylene diamine tetraacetic acid, Na₄ salt | 52.0 | g |
| potassium hydroxide, D = 1.50 | 15 | ml |

the solution was diluted with water 1:7 for use

Fixing Bath

| | | |
|---|---|---|
| ammonium thiosulfate | 130 | g |
| sodium disulfite | 10 | g |
| sodium acetate | 9 | g |
| acetic acid, 80 wt.-% | 5.6 | ml |

filled up with water to 1 liter, pH 5.4.

The sample was subsequently flushed for 10 minutes with distilled water at 40° C. to remove residual salts.

### EXAMPLE 2

### Present Invention

Developer

| | | |
|---|---|---|
| diethylene glycol | 50 | ml |
| potassium disulfite | 30 | g |
| 4-methyl-4-hydroxymethyl-l-phenyl-3-pyrazolidone | 10 | g |
| potassium hydrogen carbonate | 3 | g |
| hydroxyethane diphosphonic acid, 60 wt.-% aqueous solution | 1 | ml |
| nitrilotriacetic acid | 15 | g |
| potassium carbonate | 250 | g |
| sodium isoascorbate | 150 | g |
| potassium bromide | 15 | g |
| benzotriazole | 0.2 | g |

Filled up with water to 1 liter, pH 10.75

For usage, the solution is diluted 1:5 with water. After the dilution, the pH value is 10.5.

Fixing Bath

| | | |
|---|---|---|
| ammonium thiosulfate | 130 | g |
| sodium disulfite | 10 | g |
| sodium acetate | 9 | g |
| acetic acid, 80 wt.-% | 5.6 | ml |

Filled up with water to 1 liter, pH 5.4.

The sample was subsequently flushed for 10 minutes with distilled water at 40° C. to remove residual salts.

For comparison, a pure gelatin layer having the same application in regard to gelatin is measured, the sample length is 1 cm, the measured values are specified in S/cm. For comparison: silver: 6.2*10⁵ S/cm.

| Sample | Measured value in S/cm |
|---|---|
| Gelatin | 6*10⁻¹² |
| Example 1 (comparison) | 3*10⁻⁸ |
| Example 2 (present invention) | 7*10⁻² |

The composite material 301 shown in FIG. 4 has a middle layer 302, an upper layer 303, and a lower layer 304. The composite material 301 is applied to a carrier material 305 using its lower layer 304. In the forward area 306, the lower area 304 is already somewhat detached from the carrier material 305. Carbon fullerenes 340 are incorporated in the middle layer 302 in this exemplary embodiment.

In the following, advantageous areas of application of the present composite material 301 are explained for exemplary purposes.

### EXAMPLE 1

### Formulation and Production of NCF Compounds for Optical Reflection and Diffusion Layers for Spatial Image Representation

An optical acrylate casting resin system for use in special composite panes for heat and UV insulation in facade areas, partition and presentation surfaces, inter alia (methyl methacrylate, n-butyl acrylate, 2-ethyl hexacrylate, 2-ethyl hexyl methacrylate, boiling range>100.0° C., vapor pressure approximately 47.0-53.0 hPa), was homogeneously dispersed under high-energy ultrasound (1.7-3.5 hours, less than 50° C.) with a nanocompound (nanodiamond modification 2—0.025/1.0 wt.-% and a hydrophobic solid Aerosil/R972 −1.0/3.0 wt.-% as well as transparency-improving blue pigment −0.01/0.1 wt.-%) and stabilized.

The acrylate casting resin system is preferably cast using a cascade casting machine on triacetate or polycarbonate film, a gelatin layer also being applied for reasons of adhesion.

Two layer structure examples are described in the following.

Layer Structure 1:

| | Layer thickness wet |
|---|---|
| Gelatin (10%) | 10 μm |
| Casting solution 1 | 20 μm |

dry layer thickness: 13.8 μm

Layer Structure 2:

| | Layer thickness wet |
|---|---|
| Gelatin (10%) | 10 μm |
| Casting solution 1 | 10 μm |

dry layer thickness: 6.4 μm

Particularly favorable properties are achieved by four-layered structures.

Layer Structure 3:

| | Layer thickness wet |
|---|---|
| Gelatin (10%) | 30 μm |
| Casting solution 1 | 60 μm |
| Casting solution 2 | 60 μm |
| Casting solution 3 | 60 μm |

dry layer thickness: 158 μm

Layer Structure 4:

| | Layer thickness wet |
|---|---|
| Gelatin (10%) | 30 μm |
| Casting solution 1 | 60 μm |
| Casting solution 2 | 60 μm |
| Gelatin (10%) | 30 μm |

dry layer thickness: 92.04 μm

### EXAMPLE 2

### Production and Use of Multifunctional NCF Compounds Combined with Nanoparticles for Improving the Mechanical Properties of Lacquer Layers (Coatings) in the Example of a 2K-PUR Matte Lacquer System

Finished lacquer systems are indirectly modified using NCF particles by first pre-dispersing the nanoparticles in a solvent which is as polar and low viscosity as possible, which is already a component of the lacquer. This pre-dispersoid is used for modifying lacquer systems.

An n-butyl acetate pre-dispersoid is used for modifying the 2K-PUR matte lacquer, in which 10% monocrystalline NCF particles and 2% of the dispersing agent Disperbyk-2150 (solution of a block copolymer with basic pigment-affinity groups) are contained. The monocrystalline particles are first dispersed in an ultrasonic bath (2×600 W/Per, 35 kHz) and subsequently using an ultrasonic continuous flow apparatus (high-frequency output power 200 W, 20 kHz). To remove any contaminants, a screen having a mesh width of 65 μm is used.

500 g of the 2K-PUR lacquer (component 1) is first provided in a beaker, and subsequently admixed with 100 g sub-μm glass flakes (glass flakes made of borosilicate glass, mean size 15 μm) and 15 g nanoparticulate Aerosil® R972 (hydrophobized, pyrogenic SiO₂, mean size of the primary particles 16 nm). The additives are dispersed in the ultrasonic bath—here: glass flakes 30 minutes and Aerosil® R972 60 minutes. Subsequently, 5 g of the n-butyl acetate pre-dispersoid is stirred in, and the solution is again homogenized in the ultrasonic bath for 60 minutes. The finished nanocompound results in corresponding multifunctional improvements of the complex mechanical characteristic and performance data of the matte lacquer system.

The modified lacquer is applied (enabled) in accordance with the manufacture specifications, by mixing the modified component 1 with the prescribed quantity of hardener (component 2).

Particularly favorable properties are achieved if the monocrystalline NCF particles and 2% of the dispersing agent Disperbyk-2150 (solution of a block copolymer with basic pigment-affinity groups) is applied using a cascade casting machine to a substrate and this film is applied to the lacquer as a protector.

Inter alia, comparative changes of the surface textures were tested (matte lacquers have such an irregular fine structure of the surface that the light is scattered in all directions, and hardly any mirror effect is present), the complex mechanical characteristics, and the cross-linking density of the modified and reference lacquer systems (unmodified).

The surface texture was evaluated after treatment with steel wool fleece (by machine), with commercially available grinding and polishing pastes under a microscope at 100-fold enlargement, and by determining the roughness values using Perthometer M4Pi from Mahr according to DIN EN ISO 4287.

The ascertained roughness values—in particular the mean roughness value Rₐ—indicate significant improvement of the abrasion resistance and the Martens hardness of the modified lacquer. The texture (dullness) of the lacquer surface is subject to no or only insignificant changes in comparison to the reference lacquer after the mechanical strains. These results were confirmed by the microscopic evaluations.

Improvements were achieved in the abrasion resistance and the surface texture of the NCF-modified lacquer system, the increase of the Martens hardness values, and the improvement of the chafing resistance.

Hardness in N/mm² 228 346 (+52%)

base lacquer NCF-modified lacquer

Martens Hardness in Comparison:

| | | | | | |
|---|---|---|---|---|---|
| Gloss | 22 | 19 | 50 | 41 | base lacquer |
| degree | 5 | 6 | 11 | 13 | NCF-modified lacquer |
| in ° | before | after | before | after | |
| | angle | geom- | angle | geom- | |
| | | etry | | etry | |
| | | | | 60° | |

Abrasion Resistances in Comparison:

### EXAMPLE 3

### Production and Use of Multifunctional NCF Compounds Combined with Nanoparticles to Improve the Tribological Properties (Friction Properties) of Friction lacquer systems (dry lubricants) in the example of an NCF-modified acrylate lacquer based on water.

Finished lacquer systems are indirectly modified using NCF particles by first pre-dispersing the nanoparticles in a solvent which is as polar and low viscosity as possible, which is already a component of the lacquer. This pre-dispersoid is subsequently used for modifying lacquer systems.

The acrylate lacquer is composed of two components. Component 1 contains, inter alia, the acrylic component (Mowilith), which is very shear-sensitive. For this reason, the second component is modified here, whose components essentially only function for viscosity adjustment (thickener).

Mixture ratio: component 1—86.4 parts; component 2—13.6 parts.

To modify the component 2, an aqueous pre-dispersoid is used, which contains 5% monocrystalline NCF particles. The monocrystalline particles are first dispersed in an ultrasonic bath (2×600 W/Per, 35 kHz) and subsequently using an ultrasonic continuous flow apparatus (high-frequency output power 200 W, 20 kHz). To remove any contaminants, a screen having a mesh width of 38 μm is used.

15.3 g of component 2 is admixed with 200 g of the aqueous pre-dispersoid, and 75% of the water is removed by raising the temperature to 100° to adjust the viscosity.

The modified component 2 is subsequently stirred into 85 g of component 1. For homogenization and stabilization, the modified lacquer is treated for 30 minutes in the ultrasonic bath, admixed with 1.8 g Tamol® NN 8906 (naphthalene sulfonic acid condensation product) and dispersed again for 30 minutes in the ultrasonic bath.

Any contaminants are removed using a screen of mesh width 180 μm. The finished modified lacquer contains 6.5 wt.-% NCF particles and 1.3 wt.-% Tamol® NN 8906. The acrylate lacquer is preferably cast using a cascade casting machine on triacetate or polycarbonate film, a gelatin layer also being applied for reasons of adhesion.

Two layer structure examples are described in the following.

Layer Structure 1:

| | Layer thickness wet |
|---|---|
| Gelatin (10%) | 10 μm |
| Casting solution 1 | 20 μm |

dry layer thickness: 13.8 μm

Layer Structure 2:

| | Layer thickness wet |
|---|---|
| Gelatin (10%) | 10 μm |
| Casting solution 1 | 10 μm |

dry layer thickness: 6.4 μm

Due to the modification, the sliding friction values improved by more than double in comparison to the unmodified lacquer, the good abrasion resistances (Taber abraser test) of the acrylate lacquer being maintained. In comparison to commercial PTFE and MoS₂ friction lacquer systems, the NCF-modified acrylate lacquer has a slight improvement in regard to the sliding friction values, the abrasion resistance increasing by a factor of 6 on average here.

This is a product advantage which is reflected for the user, upon use of friction lacquers for dry lubrication, above all in increased long-term and service life lubrication and cost-effective value increase.

| | | | | |
|---|---|---|---|---|
| Abrasion | 0.18 | 0.19 | 0.34 | 0.16 |
| in %/sliding | | | | |
| friction | | | | |
| coefficient | | | | |
| sliding | 0.25 PTFE | 0.13 MoS₂ | 0.03 acrylate | 0.03 abrasion |
| friction | lacquer | lacquer | lacquer | NCF-modified |
| | | | | acrylate lacquer |

### EXAMPLE 4

### Production and Use of an Aqueous Nanosuspension (Nanocompound) for Ultra Precision Polishing On the Basis of Poly-NCF in the Example of the Grain Size Range 0-0.5 μM for High-Technology Applications Using a Carrier Pad (Base Material: Water-Based Polyacrylate)

To produce this nanosuspension, an approximately 2%, pH neutral base suspension is used as the precursor stage, which is diluted to approximately 1.5% and adjusted to pH 8 using diluted sodium hydroxide for the special application. The base suspension is composed of the poly-NCF system (0-0.5 μm), distilled water, and the stabilizers and consistency regulators polyvinyl pyrollidone (PVP or Polyvidone 25 (LAB) and nanoparticulate Aerosil® A300 (pyrogenic SiO₂, mean size of the primary particles 7 nm).

To produce the suspension, according to the present invention 100 g of the poly-NCF particles are stirred in portions into 5 kg water and first dispersed for 3 hours in an ultrasonic bath (2×600 watts/Per, 35 kHz). For further dispersion, the dispersoid is subsequently treated for 45 minutes using an ultrasonic continuous flow apparatus (high-frequency output power 1000 W, 40 kHz). Any contaminants are separated using a screen of mesh width 38 μm.

The stabilization is performed by adding 250 g Aerosil® A300 and 10 g of a 5% aqueous PVP solution. Subsequently, the batch is again dispersed for 45 minutes using the ultrasonic continuous flow apparatus.

The special pH 8 suspension is produced—batch approximately 4.8 kg—by deluding 3.6 kg of the base suspension with 1.2 kg distilled water (in the ratio 3:1, w:w) and subsequently homogenizing it in the ultrasonic bath for 15 minutes. The pH value of the suspension is adjusted to pH 8±0.2 using 1.5% sodium hydroxide. The standard value is approximately 9±2 ml diluted sodium hydroxide/kg suspension.

Composition of the Nanocompound in wt.-%:

| | |
|---|---|
| Poly-NCF (0-0.5 μm): | 1.4% |
| distilled water: | 95.0% |
| Aerosil ® A300: | 3.6% |
| Polyvidone: | 0.007% |
| NaOH (s): | 0.012 ± 0.004% |

### EXAMPLE 1

### Comparison

Preferably, the nanosuspension is cast using a cascade casting machine on triacetate or polycarbonate film, a gelatin layer also being applied for reasons of adhesion.

Two layer structure examples are described in the following.

Layer Structure 1:

| | Layer thickness wet |
|---|---|
| Gelatin (10%) | 10 μm |
| Casting solution 1 | 20 μm |

dry layer thickness: 13.8 μm

Layer Structure 2:

| | Layer thickness wet |
|---|---|
| Gelatin (10%) | 10 μm |
| Casting solution 1 | 10 μm |

dry layer thickness: 6.4 μm

Achieved test parameters and performance characteristics: area of use: ultra-precision final polish of planar special stepper optics made of CaF₂.

Test Experiments:

Experimental Preparation:

A round CaF₂ part, on which a small area is covered with protective lacquer, to thus preserve the state before etching/dissolving attack of the polishing agent.

Experimental Sequence:

The previously specified special optic is processed according to a standard method on one half using a rotating tool, covered with a soft polishing cloth, to achieve constant removal. Processing is performed in perpendicular meandering paths beginning from the left edge. Approximately ½ liter of the suspension is added as it rotates. Processing times are between 30 minutes and 5 hours. Experiments using competitive standard products are performed in the same sequence described as the comparative basis.

Results:

Mean removal using this novel suspension 800 nm (comparison to standard D 0.25 suspensions: 300 to 500 nm).

Achieved Micro Roughnesses:

at 2.5x=1.1 to 1.2 nm (comparison to standard D 0.25=1.3 to 1.7 nm) at 20x=0.6 to 0.7 nm (comparison to standard D 0.25=1.1 to 1.7 nm)

Scratch Status:

With these novel systems (measured in the dark field microscope—200-fold enlargement) no visible scratches (comparison to standard D 0.25=clear and multiple significant scratches visible).

[see source for figure]

dark field microscopic pictures 200 fold enlargement, left carbo-tec (scratches do not result in contrast in video printer) right, same processing using standard D 0.25 for comparison (background scratching distributed uniformly over area)

### EXAMPLE 5

### Nanocompound Based on NCF for Heat Management (Heat Conduction Films and Layers)

The use of new generations of components of power electronics as well as the implementation of innovative system integrations with growing miniaturization and increase of the processing speeds, predominantly in the fields of motive control and regulation, requires, inter alia, the use of heat conduction systems partially having increased requirements for the parameter and performance characteristics.

Following table 1 shows selected and currently desired framework parameters.

In particular the targeted expansion of the thermoconductive bandwidth in the range of over 5 W/mk (up to 20 W/mk), makes the development of innovative heat-conducting filler systems necessary, which ensure optimum heat sink and above all heat transfer effects at the corresponding phase and contact boundaries at elevated operational temperatures of the overall complex.

The condition required for this purpose is: the formulation of specially structured and combined material composites and their targeted introduction (enabling) into the particular overall system of the carrier matrix.

TABLE 1
desired material properties
Heat-conducting high flexibility adhesives

| Property | Prior art | Desired |
|---|---|---|
| Application | Cooling body | Heat sink attach. |
| Substrate | Al, Cu | Al, Cu |
| Components | LTCC (low | LTCC, DPC (low |
| | temperature | temperature |
| | cofired ceramics) | cofired ceramics, |
| Geometry | Up to 2 inches | Up to 2 inches |
| Coupling method | n.a. | n.a. |
| Number of | n.a. | n.a. |
| couplings | | |
| Processing | Pin transfer | Pin transfer |
| Viscosity at room | | <100 Pa @ D = 1 s⁻¹, |
| temperature | | non-thixotropic |
| Desired working | | >3 days |
| life | | |
| Desired curing | | <30 minutes at |
| time | | 150° C. |
| Dielectric | >20 kV/mm | >20 kV/mm |
| strength | | |
| Electrical | k.A. | k.A. |
| conductivity | | |
| Thermal | (2-3) W/mK | (5-20) W/mK |
| conductivity | | |
| Load current | k.A. | k.A. |
| CTE | | |
| Glass transition | <−45° C. | <−45° C. |
| temperature | | |
| Modulus | | |
| Ductile yield | 30% | >70% |
| Adhesion force | >2 N/mm² | >2 N/mm² |
| Operating | Up to 150° C. | >165° C. |
| temperature | | |
| Working time | | |
| Environmental | −40° C./+150° C. | −40° C./+165° C. |
| conditions | | |
| (climate) | | |
| Mechanical | | 40 g (vibration) |
| conditions | | |

Two formulations selected as examples are observed as matrix systems, based on cycloaliphatic epoxide (system a) and on polymer silicones (system b) (abstract 1), which require different technological implementation approaches.

Preferably, the NCF solution is cast using a cascade casting machine on triacetate or polycarbonate film, a gelatin layer also being applied for reasons of adhesion.

Two layer structure examples are described in the following.

Layer Structure 1:

| | Layer thickness wet |
|---|---|
| Gelatin (10%) | 10 μm |
| Casting solution 1 | 20 μm |

dry layer thickness: 13.8 μm

Layer Structure 2:

| | Layer thickness wet |
|---|---|
| Gelatin (10%) | 10 μm |
| Casting solution 1 | 10 μm |

dry layer thickness: 6.4 μm

Excerpt 1: requirements for thermally conducting filler in view of the formulation.

The finished system is to be highly flexible.

Therefore, the filler cannot increase the rigidity of the system. The ionic component of the finished formulation is to be as small as possible. Various base polymers must be tested. Therefore, the filler mixtures must be compatible with the following organic compounds.

System a: this system is based on a cycloaliphatic epoxide. The chemical formula is shown in the following. This resin has a viscosity of 300 mPa s and a density of 1.16 g/cm³. It begins to polymerize under the influence of acids or bases.

[see source for formula]

The filler mixture may be a dry powder or a mixture of the filler and this resin. Up to 80 wt.-% other organic compounds are added to the system. The proportion of the filler is to be as high as possible in this mixture, otherwise the danger exists that the mixture may not be varied in the required range.

System b: various polymers based on silicone are used as the flexible base polymer. Because the properties and curing conditions of silicones may only be varied via different molecular weights and the type/number of the functional groups, it is advantageous if the filler is a dry powder. This powder is preferably compatible with silicone oil (poly(dimethyl siloxane), PDMS).

Liquid mixtures are rather disadvantageous, and usually not possible, because it would require a separate mixture for each of the numerous reactive silicones to be tested. In the present case, a method is required, using which the dry filler may be distributed in the silicone mixture.

Formulation of Optimized Heat Conduction Compounds (Variations):

Multicomponent use of morphologically different heat conduction fillers in the composite with highly structured nanomonosystems and nanopolysystems (NCF) for staggered formulation of the desired optimal thermoconductive characteristics and to ensure the required cost effectiveness at high fill levels.

Implementation of optimized combinations of filler particle sizes in broadband size distribution of 10/50 nm to 15/23 μm having multiple overlapping distribution maxima (peaks). Build up of compound formulations and combination of spherical, dendritic, fibrous, and/or lamellar filler particles and cluster forms. Activation (doping) of the required surface activities, predominantly of the highly structured nanocarbon fullerenes used in the compound.

Adaptation of the Enabling Technology for Compounding the Carrier Matrix:

Compound design for system a: stable monomeric dispersion.

Compound design for system b: pre-formulated dry dispersoid in homogeneous form.

1. A composite material made of at least three layers, wherein at least one of the layers has active ingredients, ceramic nanoparticles, silver salts, or nanoparticulate carbon modifications.

2. The composite material according to claim 1, wherein at least one of the layers (2, 3, 4) has medicinal active ingredients (7) or bitter principles.

3. The composite material according to claim 1, wherein at least one of the layers (2, 3, 4) has sensitized silver salts (230).

4. The composite material according to claim 1, wherein at least one of the layers (2, 3, 4) has carbon fullerenes (340).

5. The composite material according to claim 1, wherein at least one of the layers (2, 3, 4) of the composite material (1) has a layer thickness having a tolerance of less than ±10%, preferably having a tolerance of ±1%.

6. The composite material according to claim 1, wherein the layer (2), which has at least one component, namely active ingredients, in particular medicinal active ingredients (7) or bitter principles, ceramic nanoparticles (120), silver salts, in particular sensitized silver salts (230), or nanoparticulate carbon modifications, in particular carbon fullerenes (340), is enclosed by at least two further layers (3, 4).

7. The composite material according to claim 1, wherein at least one of the layers (2) has first components (7) which are different from further components (8) of further layers (3, 4).

8. The composite material according to claim 1, wherein at least two of the layers (3, 4) have identical components (8).

9. The composite material according to claim 1, wherein at least two of the layers (3, 4) have identical properties.

10. The composite material according to claim 1, wherein at least one of the layers (2, 3, 4) has a layer thickness of less than 20 μm or less than 0.5 μm.

11. The composite material according to claim 1, wherein at least one of the layers (2, 3, 4) has a layer thickness of more than 0.1 μm or more than 10 μm.

12. The composite material according to claim 1, wherein at least two of the layers (2, 3, 4) have different layer thicknesses from one another.

13. The composite material according to claim 1, wherein at least one of the layers (2, 3, 4) has a ceramic suspension having less than 80 wt.-%, preferably less than 70 wt.-% ceramic nanoparticles (120).

14. The composite material according to claim 1, wherein at least one of the layers (2, 3, 4) has a ceramic suspension having more than 30 wt.-%, preferably more than 45 wt.-% ceramic nanoparticles (120).

15. The composite material according to claim 1, wherein the ceramic nanoparticles (120) have a grain size of less than 5000 nm, preferably less than 1000 nm.

16. The composite material according to claim 1, wherein the ceramic nanoparticles (120) have a grain size of more than 10 nm, preferably more than 100 nm.

17. The composite material according to claim 1, wherein at least one of layers (2, 3, 4) has flavoring substances (8).

18. The composite material according to claim 1, wherein at least one of the layers (2, 3, 4) has gelatin.

19. The composite material according to claim 1, wherein at least one of the layers (2, 3, 4) has a thickener, a hardening agent for a component of a layer (2, 3, 4), such as gelatin, or a cross-linking agent for a component of a layer (2, 3, 4), such as gelatin.

20. The composite material according to claim 1, wherein at least one of the layers (2, 3, 4) is electrically conductive.

21. The composite material according to claim 20, wherein the electrically conductive layer (102) has silver and/or silver salt.

22. The composite material according to claim 20, wherein the electrically conductive layer (102) is enclosed by electrically insulating layers (103, 104).

23. The composite material according to claim 1, comprising an electrically insulating layer (103, 104), which has a polymer, such as gelatin, as an insulator.

24. The composite material according to claim 1, wherein one of the layers (102) is an electrically functionalized film.

25. The composite material according to claim 1, wherein the silver salts are sensitized in a range above 300 nm, preferably above 350 nm.

26. The composite material according to claim 1, wherein the silver salts are sensitized in a range below 800 nm, preferably below 750 nm.

27. The composite material according to claim 1, wherein the silver salts are fixed using ammonium thiosulfate or sodium thiosulfate.

28. The composite material according to claim 1, wherein fullerenes, such as carbon fullerenes (340), are embedded in a polymer.

29. The composite material according to claim 1, wherein layers containing polymer comprise a water-soluble polymer.

30. The composite material according to claim 1, wherein at least one of the layers (2, 3, 4) is situated on a carrier material (5).

31. The composite material according to claim 1, wherein the composite material (1) is implemented symmetrically.

32. The composite material according to claim 1, wherein the composite material (1) has a cylindrical body.

33. The composite material according to claim 1, wherein the composite material (1) comprises a film, particularly a ceramic film.

34. A composite material made of at least three layers, wherein the composite material is produced using a cascade casting machine or a curtain casting machine.

35. The composite material according to claim 34, wherein the composite material (1) has at least one of the layers having active ingredients, ceramic nanoparticles, silver salts, or nanoparticulate carbon modifications.

36. A method for producing a composite material having at least three layers, in which the layers are cast on a carrier material, wherein at least one further layer admixed with a component different from the first component is cast onto a layer admixed with the first component.

37. The method according to claim 36, wherein at least three layers (2, 3, 4) are cast onto the carrier material (5) in one work step.

38. The method according to claim 36, wherein a layer (2) admixed with active ingredients, in particular with medicinal active ingredients (7) or with bitter principles, has at least two further layers (3, 4), which are different from the first layer (2), cast around it.

39. The method according to claim 36, wherein a layer (102) admixed with a ceramic suspension, in particular with ceramic nanoparticles (120), has at least two further layers (103, 104), which are different from the first layer (102), cast around it.

40. The method according to claim 36, wherein a layer (202) admixed with sensitized silver salts (230), has at least two further layers (203, 204), which are different from the first layer (202), cast around it.

41. The method according to claim 36, wherein a layer (302) admixed with nanoparticulate carbon compounds, in particular with carbon fluorine (340), has at least two further layers (303, 304), which are different from the first layer (302), cast around it.
